(19) United States
(12) Patent Application Publication
(10) Pub. No.: US 2007/0167847 A1
(43) Pub. Date: Jul. 19, 2007

(54) Method and device for using a physiological parameter to express evolution

(76) Inventor: Michael F. Guglielmino, Woodland Hills, CA (US)

Correspondence Address: KOLISCH HARTWELL, P.C., 200 PACIFIC BUILDING, 520 SW YAMHILL STREET, PORTLAND, OR, 97204, US

(21) Appl. No.: 11/337,192

(22) Filed: Jan. 19, 2006

(51) Int. Cl. A61B 5/04

(52) U.S. Cl. 600/509

(57) ABSTRACT

A method for presenting a bioorganism's evolutionary passage through at least a portion of its life, by reference to a selected physiological parameter emanating from the bioorganism, as an alternative to use of conventional horology, which includes selecting a physiological parameter specific to the bioorganism itself, one which continuously and repeatedly occurs, for the most part involuntarily, throughout and only throughout the bioorganism's life, and then detecting the repeated occurrences generated by the parameter.

### BACKGROUND OF INVENTION

The present invention is directed to a method and device used to measure the evolution of a bioorganism over its life or a portion thereof, without reference to time, or reference to conventional horological systems. Time has been measured over the centuries by conventional systems which are related to movement of this planet, one day being measured by the earth making a single revolution about its axis. Conventional horology divides time into successive units, i.e., seconds, 60 seconds per minute, 60 minutes to an hour, 24 hours in a day, and so forth.

With respect to a bioorganism, such as a human being, such a conventional measure of time, as an indicator of a person's life, is essentially irrelevant at least from the standpoint of how an individual's body itself physically is evolving or expressing itself. While a person's life may be chronologically measured by how many years they live, or how old they are at any given time, rotation of the earth about its axis is not an expression of the body itself, and how the body actually is evolving, or how it is undergoing changes, either at a given moment or over a given period in one's life. Human beings, in common with all bioorganisms in the mammalian class, live and function under a complex physiological system which includes various parameters, such as a beating heart, respiration, body temperature regulation, ingestion, digestion, blood pressure and numerous others. These parameters can be observed and measured, and the body expresses itself through them, and all are specific and unique to each individual.

There have been numerous proposals for providing so-called “biofeedback,” as measured by a person's “biorhythms,” and other devices have been developed to detect pulse, commonly employed in a jogging watch, a stopwatch or wristwatch.

For example, a conventional pulse counter for electronically counting the number of pulses and displaying the count is shown in U.S. Pat. No. 4,009,708, which describes a wristwatch type pulse counter capable of counting the number of pulses per minute.

In U.S. Pat. No. 4,101,071, there is described an apparatus for calculating a calorie bum total according to the number of pulses and the length of exercise time. An assembly obtained by incorporating a pulse sensor in an electronic wristwatch is also known. For example, U.S. Pat. No. 3,937,004 describes a technique for incorporating a pulse sensor in a wristwatch. U.S. Pat. No. 4,086,916 describes a technique for incorporating a pulse sensor in a wristwatch band.

In addition, U.S. Pat. No. 3,978,849 describes an apparatus for displaying an optimal exercise amount as well as the number of pulses, or signaling to the user that the number of pulses is too high.

### SUMMARY OF THE INVENTION

It is proposed by the present invention that a more meaningful method for a person to view their evolution is not through measurement of time, but through measurement of a selected physiological parameter or measurement of multiple parameters. One physical expression or parameter which could be effectively utilized is a cardiac signal, or more simply, a person's pulse as measured by a series of heartbeats. A human being likely has a certain, albeit unknown, ultimate number of heartbeats unique to their own life, assuming no premature death brought on by accident or disease.

A heartbeat, or measured series of heartbeats, is a physical expression emanating from an organism, and the rate of heartbeats can and does change, depending on factors such a gender, one's stage in life, level of physical activity, emotional state, health, blood pressure, and certainly other things. As such, a physical expression, such as a heartbeat, when viewed progressively, gives a personal or specific reference to how an individual person is evolving during their life, how their body is responding.

Broadly, then, the present invention can be summarized as a method for presenting a bioorganism's evolutionary passage through at least a portion of its life, by reference to a selected physiological parameter emanating from the bioorganism, as an alternative to use of conventional horology, which includes selecting a physiological parameter specific to the bioorganism itself, one which continuously and repeatedly occurs, for the most part involuntarily, throughout and only throughout the bioorganism's life, and then detecting the repeated occurrences generated by the parameter. But it is more than just detection; the occurrences are arranged or grouped into basic units, and these are displayed and recorded so that the bioorganism's evolving life or segments thereof, over time, can be measured and interpreted by reference to the recorded units, and groups thereof, as an alternative to using horology as a measure.

Human beings experience a resting pulse that can range, depending on health, age, physical condition, gender, etc. from about 45 to 90 beats per minute. Of course the pulse can be much higher depending upon level of physical activity, emotional state and other factors. The point is that an average heartbeat may be in the range of 65-85 beats per minute, say, 75 beats per minute, as a normal resting pulse. This corresponds somewhat roughly to the number of seconds in a minute, which is of course, 60. Thus, if a person's heartbeats were measured in a method or system which recorded each heartbeat as a unit, then those heartbeats could be presented on a display device or monitor, such as a face similar to that of a wristwatch.

These units can be grouped, and conventions assigned to them, so they could be observed, much like one observes a clock, but in this case what is being observed is a person's ongoing physical experience, as represented by the physiological parameter. A wristwatch-type device could be utilized to have either the display face or wristband provided with a pulse sensor to record the pulse at the wrist, but measurements could be made at the external situs of the carotid or femoral arteries. This device would use the body's physical expression, as embodied in heartbeats, durationally displayed, over a selected time period, to present to a person their own “natural time,” one's own natural rhythm or tempo which would be the guide, and not the rotation of the earth.

### BRIEF DESCRIPTION OF THE DRAWINGS

FIG. 1 is a view of a person's hand and wrist showing a monitor and a pulse sensor for use mounted in position, like a wristwatch, for recording heartbeats;

FIG. 2 is an enlarged view of a monitor, such a dial face marked with a scale showing a single sweep hand and a scale for displaying heartbeats and recording them;

FIG. 3 is another view, of a digital monitor, illustrating how the heartbeats could be continuously displayed; and

FIG. 4 is a view of a person with a band mounted on the chest for sensing rate of respiration, to illustrate another example of how a different physiological parameter could be employed to record a physical experience.

### DETAILED DESCRIPTION OF THE PREFERRED EMBODIMENTS

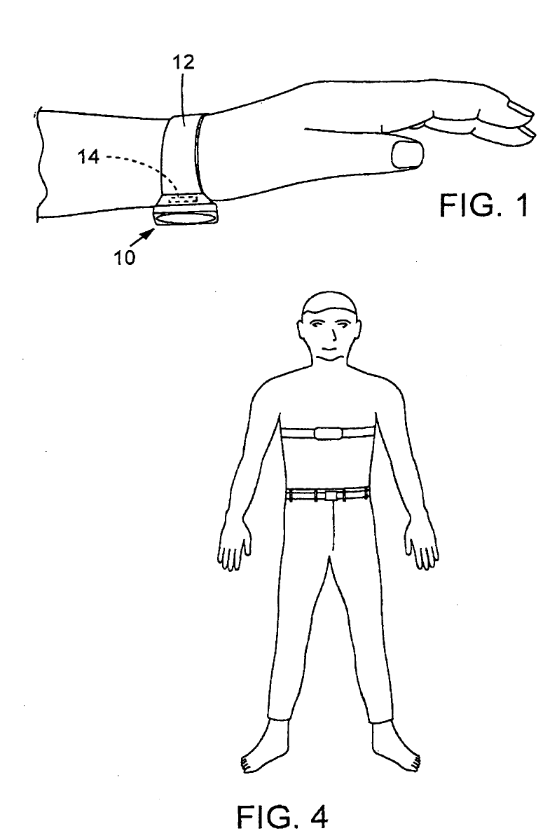

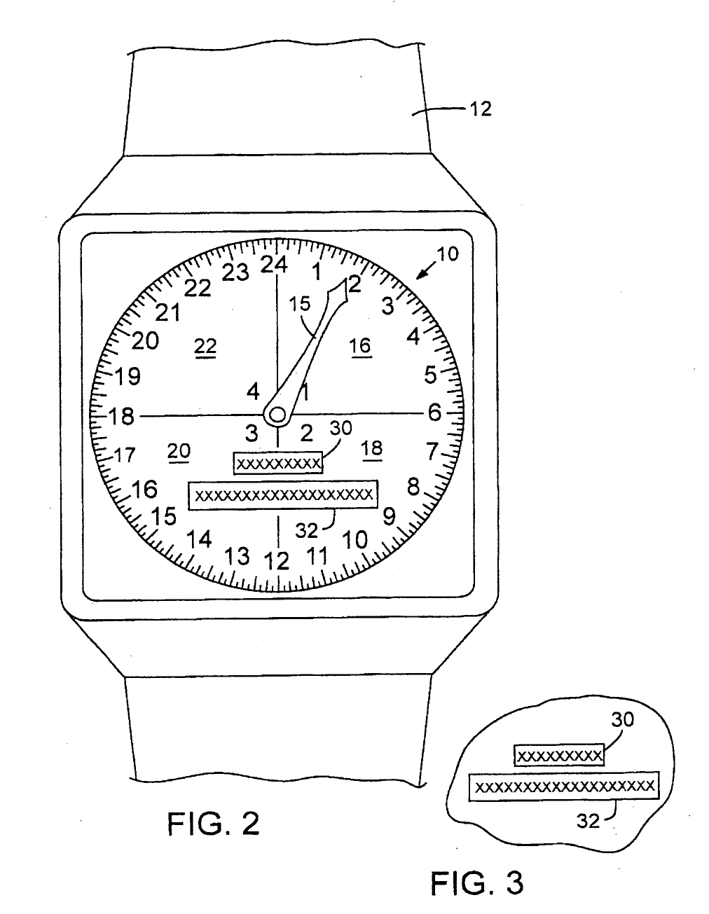

As shown in FIGS. 1 and 2, a monitor, in the form of a wristwatch-like display face, is utilized so that the sequence of each heartbeat could be shown by a dial or hand which sweeps along from heartbeat to heartbeat, measured in increments on a scale on the dial face. There are different ways in which a scale could be created, for example, assume that a person knew their resting pulse averaged 75 beats per minute. A monitor could be made with marks on a dial face, as shown in FIG. 2. Here, a dial face, indicated at 10, is mounted on a wristband 12, and a sensor 14 provided on the wristband is mounted to record each heartbeat. The sensor is conventional, as known in the art, to record a pulse, but in this case is used to record pulses which can be displayed in a unique convention.

For example, a single heartbeat may be thought of as an “instantaneous experience” or “lex.” Using such a convention, after 75 heartbeats×60, or 4,500 heartbeats have occurred, it is deemed that a so-called “long experience” or 1 “Lex” has taken place, somewhat analogous to an hour, and this “Lex” marker is pointed out by a sweep hand 15, the first “Lex” marker being shown as the number “1” on the dial face, in the embodiment shown in FIG. 2.

As also shown, the dial face in this embodiment is divided into quadrants, such as indicated at 16, 18, 20 and 22, and each quadrant includes six “Lex” markers or indicia, each of which notes that 75×60 or 4,500 heartbeats have occurred.

The “Lex” marker denoted “2,” confirms that 9,000 heartbeats have taken place. FIG. 2 also shows that the scale between each “Lex” is divided into marks, each of which represents about 562 heartbeats. To provide a scale which is marked to show each heartbeat, in the convention shown here, would be difficult because of the sheer number of heartbeats. Suffice it to say that different scales could be chosen, and the device calibrated to show the “Lex” as described above is representations of one way; certainly a different scale could be chosen. The point is, by the scale arrangement shown, hand 15 will sweep all the way around during some period which may be thought of as something roughly equivalent to day, assuming 75 heartbeats per minute. Of course, the real rate of heartbeats may be very different, and using the convention shown in FIG. 2, sweep hand 15 may move much faster, depending on level of physical activity, for example. A person may have several periods of high physical activity or emotional extremes which will cause the hand to complete a single revolution very differently from one day to the next. Whatever the result, a person will see their own “experience” differ from day to day, measured by their own heartbeat, in this example.

The idea here is that one complete sweep of the dial or sweep hand 15, will complete roughly one day, although it is not exactly a day, but rather a person's physical expression of what occurs, or would normally occur, if the person were at rest. In this instance, the “Lex” indicia marked at 1, 2, 3, 4, etc., are noted by the sweep hand, as the day progresses. The dial face has been divided into the four quadrants, in this case, because there is only a single dial or sweep hand which is powered (by a battery synchronized with the heartbeat) to move. The dial starts at the top position, and ends at the indicia mark 24. Two hands or dials could be used, like the hour and minute hands on a clock or watch. Thus, one hand would be continuously displaying the “lex,” and the other would point to the “Lex.”

It is to be noted that the dial face is also provided with a display window 30 which displays in digital format, the total number of heartbeats during a wearing sequence (assuming the device is taken off at some time) and a perpetual memory, shown at 32, also a digital display, which shows the total heartbeats accumulated during the period the device has been worn. Other modifications could be built into the device, for example, memory could keep the device working, when not worn, so that a preprogrammed resting pulse will continuously be recorded; when the person puts the device back on, activates it appropriately, the real-time pulse is noted, which may or may not correspond to the average.

Again, assuming that an average heart beats at a rate of 75 per minute, the total heartbeats in a 24 hour day will be approximately 106,000, computed as follows:
75 beats/min.×60 min./hr×24 hrs=106,000 heartbeats.

This is assuming of course, a steady 75 beats per minute which probably is unlikely. Too many events can happen in a typical day, including exercise, stress, eating, etc. which will change the number of heartbeats per minute.

But the point here is that by using heartbeats, one can be acting on their own “natural time,” where time is expressed by some physical parameter unique to an individual. This system can be broken down as follows:
1 lex=1 heartbeat
1 Lex=approx. 4,500 lex
1 quad=6 Lex=27,000 Iex
24 Lex in a day=108,000 Iex or 108,000 heartbeats

At any given time, a person can look at the monitor and know what their natural rate of evolution is, and utilize this to gauge and monitor their own activities. For example, a person may choose to do something according to their own physical time, as expressed through their heartbeats. The monitor could be a completely digitized device, with a digital display, as shown in FIG. 3. In that figure, the face of the device, indicated at 30, includes windows which digitally display the “lex” (total number of heartbeats), the “Lex” (4,500 heartbeats per “Lex). The bottom window or panel shows in digital format the total “lex” and total “Lex.” The upper window could be reset everyday or whenever the person desires.

It is to be noted that display window 30 could be a digital counter or display which continuously records the heartbeats and may be reset whenever a person wants. This display of continuous “lex” will eventually be a very large number, ranging into the billions of heartbeats. The lower display 32, may record the total number of heartbeats over time, and will be stored in memory. Obviously, over time, the lower display may be a number ranging well into the billions.

Moreover, the device could be programmed so that the device keeps recording the average beats per minute, thus if the device is removed, a person's evolution, using heartbeats is still being recorded, based on the known, preprogrammed average number of beats per minute.

The display windows could also be configured so that the upper window displays only “Iex” or a combination of “lex” and “Lex.” Thus, the upper window may show a number like 99,000, which would mean that 24 “Lex” are close to being reached. The bottom windows could be divided into sections which show “lex” and “Lex,” or just include “lex” which, over time, will become a very high number, into the billions.

The method and device of the present invention can also be utilized so that a person could see specifically how their “lex,” as it actually occurs, relates to their average pulse rate. This can be accomplished by using two hands; for example, one hand is driven to display the “lex” and “Lex” as they would be synchronized for 75 beats per minute, for example. The other hand would sweep at the actual rate of heartbeats; this latter or second hand may move much more rapidly or advance from “Lex” to “Lex” in advance of the first hand. A person observing this would reflect on being “ahead of time,” so to speak. They may choose to curtail activities, to try to return their pulse to its average rate, thus “preserving” their heartbeats, as it were. Conversely, a person's pulse may decrease, through sedentary or listless activity, and where this happens, the “lex” as it occurs would lag behind the first sweep hand which moves in accordance with a preprogrammed 75 beats per minute, for example.

The invention as described uses technology which is available, pulse sensors are known, and to program a sweep hand so that it moves sequentially along a dial in accordance with each heartbeat can readily be done. To provide a second hand which is timed, technology can be used which is already employed in stop watches, for example, or jogging watches. The dial face could be totally digital, if desired.

The above are just examples, the important thing to note is that a physiological parameter, i.e., a person's pulse or heartbeat, which continuously and repeatedly occur, are presented so that a person can view their own body's working over time. As shown in FIG. 4, the rate of respiration could be used as the employed physiological parameter. Here there is a sensor band placed around a person's chest, so that the rate of breathing can be displayed on a dial or watch face device, interconnected by means not shown. Other physiological parameters could be used, temperature change, perhaps, and somewhat more arbitrary ones, for example, blinking. Doubtless, there are others, but the emphasis here is that it is a physical experience, recorded and displayed, grouped in a preselected convention, which provides the guide to self-observed evolutionary passage, rather than the rotation of the earth.

I claim:

1. A method for presenting a bioorganism's evolutionary passage through at least a portion of its life, by reference to a selected physiological parameter emanating from the bioorganism, as an alternative to use of conventional horology, comprising the steps of:
selecting a physiological parameter specific to the bioorganism itself, one which continuously and repeatedly occurs, for the most part involuntarily, throughout and only throughout the bioorganism's life;
detecting the repeated occurrences generated by the parameter;
recording the occurrences successively and continuously and arranging them in basic units; and
displaying the units so that the bioorganism's evolving life or segments thereof, over time, can be measured and interpreted by reference to the recorded units, as an alternative to using horology as a measure.

2. The method of claim 1 wherein the displaying step further includes presenting the units simultaneously with each occurrence.

3. The method of claim 2 wherein the recording step includes accumulating and retaining the units so that a cumulative measure of all occurrences generated by the parameter can be selectively displayed.

4. The method of claim 3 wherein the display step includes the capability of selectively displaying the units as they occur, along with a cumulative display of all the units generated over time, or selected period.

5. The method of claim 1 wherein the displaying step is presented digitally, and is selectively viewable by the bioorganism or another.

6. The method of claim 1 wherein the selected occurrences can be regulated at least partially over a selected duration by voluntary effort of the bioorganism.

7. The method of claim 1 wherein cardiac signals generated by the bioorganism itself are selected as the physiological parameter for detecting, recording and displaying.

8. The method of claim 7 wherein the cardiac signals are represented by the bioorganism's pulse, and wherein each basic unit is defined as being a single heartbeat.

9. A method for presenting a bioorganism's evolutionary passage through at least a portion of its life, by reference to a selected physiological parameter emanating from the bioorganism, as an alternative to use of conventional horology, comprising the steps of:
selecting cardiac signals generated by the bioorganism itself as the physiological parameter;
detecting the cardiac signals generated by the bioorganism;
displaying the cardiac signals by arranging them in basic units; and
accumulating the basic units successively and continuously, so that the bioorganism's evolving life or portion thereof, over time, can be measured and interpreted by reference to the recorded basic units, or cardiac signals, as an alternative to using horology as a measure.

10. The method of claim 9 wherein the bioorganism's cardiac signals are represented by the bioorganism's pulse, and wherein each basic unit further defined as being a single heart beat.

11. The method of claim 10 wherein the displaying step further includes presenting the basic units simultaneously with each heartbeat.

12. The method of claim 11 wherein the recording step includes accumulating and retaining the basic units so that a cumulative measure of all heart beats can be selectively displayed.

13. The method of claim 12 wherein the display step includes the capability of selectively displaying the basic units as each heart beat occurs, along with a cumulative display of all the heart beats generated over time, or a selected period.

14. The method of claim 13 wherein the displaying step is presented digitally, and is selectively viewable by the bioorganism or another.

15. A method for presenting a bioorganism's evolutionary passage through at least a portion of its life, by reference to a selected physiological parameter emanating from the bioorganism, as an alternative to use of conventional horology, comprising the steps of:
selecting the rate of respiration of the bioorganism itself as the physiological parameter;
detecting the rate of respiration generated by the bioorganism;
arranging the rate of respiration in basic units for display; and
accumulating the units successively and continuously, so that the bioorganism's evolving life or portion thereof, over time, can be measured and interpreted by reference to the accumulation of the basic units or rate of respiration, as an alternative to using horology as a measure.

16. A method for presenting a bioorganism's evolutionary passage through at least a portion of its life, by reference to a selected physiological parameter emanating from the bioorganism, as an alternative to use of conventional horology, comprising the steps of:
selecting a physiological parameter specific to the bioorganism itself, one which continuously recurs, for the most part involuntarily, throughout and only throughout the bioorganism's life, but one which can be regulated at least partially over a selected duration by voluntary effort of the bioorganism;
detecting the repeated occurrences generated by parameter;
recording the occurrences successively and continuously and arranging them in basic units; and
displaying the units so that the bioorganism's evolving life or segments thereof, over time, can be measured and interpreted by reference to the recorded units, as an alternative to using horology as a measure.
